(19) United States
(12) Patent Application Publication
(10) Pub. No.: US 2003/0153073 A1
(43) Pub. Date: Aug. 14, 2003

(54) Expansion of T cells in vitro and expanded T cell populations

(76) Inventors: Paul Rogers, San Diego, CA (US); Toshifumi Mikayama, Nagareyama-shi (JP)

Correspondence Address: Pillsbury Winthrop LLP, Intellectual Property Group, Suite 200, 11682 El Camino Real, San Diego, CA, 92130-2092, US

(21) Appl. No.: 10/291,809

(22) Filed: Nov. 7, 2002

Related U.S. Application Data
(60) Provisional application No. 60/336,776, filed on Nov. 7, 2001.

(51) Int. Cl. C12N 5/08; A61K 45/00

(52) U.S. Cl. 435/372; 424/93.7

(57) ABSTRACT

Methods for expanding T cells (e.g., Vα24⁺Vβ11⁺ human NKT cells ) in vitro by repeated culturing in the presence of an antigen (e.g., glycosphingolipid), antigen presenting cells, and a cell survival factor are provided. A population of NKT cells (Vα24⁺ζβ11⁺) can be expanded more than 1 million-fold in 5 weeks and more than 1 billion-fold in 10 weeks.

### RELATED APPLICATIONS

This application claims priority to application serial No. 60/336,776, filed Nov. 7, 2001.

### TECHNICAL FIELD

The invention relates generally to immunology and more particularly to expanding T cell numbers in vitro and uses of such expanded T cell populations.

### BACKGROUND

Natural killer T (NKT) cells are a subset of CD3⁺ T cells which express cell surface receptors, such as CD161 (NKR-P1A, associated mainly with the NK cell lineage). A small subpopulation of human NKT cells (CD3⁺CD161⁺) express a highly conserved T cell receptor chain Vα24-JαQ which preferentially associates with Vβ11 (Bendelac, et al., Annu Rev Immunol 15:535 (1997); Dellabona, et al., J Exp Med 180:1171 (1994)). This Vα24⁺Vβ11⁺ T cell population has been linked to many immunological disorders.

Human Vα24⁺ NKT cells show a high degree of phenotypic and functional conservation with murine Vα14 NKT cells which suggests an important biological function in the immune system (Kawano, et al., Science 278:1626 (1997); Spada and Porcelli, J Exp Med 188:1529 (1998); Nieda, et al., Hum Immunol 60:10 (1999); Brossay, et al., J Exp Med 188:15221 (1998)). As in mouse, human Vα24⁺ or KRN7000-reactive T cells may express surface molecules such as CD161 (or NK1.1 in the mouse) which are typically associated with natural killer (NK) cells. However, this molecule as well as others are affected by activation state of the T cell and may be absent on some populations Vα24⁺ T cells or T cell lines (Takahashi, et al., J Immunol 164:4458 (2000)). Therefore, the KRN7000-reactive or Vα24⁺ T cells may not strictly be classified as NKT cells although they may exhibit NK cell-like killing activity.

Both human Vα24⁺ and mouse Vα14⁺ NKT cells are dependent on and activated by CD1d⁺ antigen presenting cells which present the glycolipid α-galactosylceramide (also known as KRN7000) (Kawano, et al., Science 278:1626 (1997); Spada and Porcelli, J Exp Med 188:1529 (1998); Nieda, et al., Hum Immunol 60:10 (1999); Brossay, et al., J Exp Med 188:15221 (1998); Burdin, et al., J Immunol 161:3271 (1998)). CD1 molecules area family of related proteins encoded by closely linked genes on chromosome 1 and have homology to both major histocompatibility (MHC) class I and class II proteins (Porcelli and Modlin, Annu Rev Immunol 17:297 (1999))). Four of the human CD1 genes (CD1a, b, c, and d) are known to be expressed as proteins and associate non-covalently with β2-microglobulin. CD1d proteins are expressed by antigen presenting cells including B cells, monocytes, and dendritic cells (Exley, et al., Immunology 100:37 (2000); Spada, et al., Eur J Immunol 30:3468 (2000)) and also by some activated T cells, cortical thymocytes, and intestinal epithelium (Exley, et al., Immunology 100:37 (2000); Blumberg, et al., J Immunol 147:2518 (1991)).

In addition to CD1d, optimal activation of human Vα24⁺ NKT cells can be obtained by presentation of KRN7000 (Nieda, et al., Hum Immunol 60:10 (1999); Brossay, et al., J Exp Med 188:15221 (1998)) or related analogs (Ishihara, et al., J Immunol 165:1659 (2000)) which bind to CD1d. The corresponding T cell population in mouse can also be activated by analogs of KRN7000 (Brossay, et al., J Immunol 161:5124 (1998)) and possibly by an endogenous phospholipid which binds to murine CD1d1 (Joyce, et al., Science 279:1541 (1998)). KRN7000 can induce rapid cytokine secretion and potent anti-tumor responses in mice (Toura, et al., J Immunol 163: 2387 (1999); Nakagawa, et al., Oncol Res 12:51 (2000); Nakagawa, et al., Cancer Res 58:1202 (1998); Morita, et al., J Med Chem 38:2176 (1995)) and can elicit killing of human tumor cell lines (Takahashi, et al., J Immunol 164:4458 (2000); Nicol, et al., Immunology 99:229 (2000); Metelitsa, et al., J Immunol 167:3114 (2001); Kawano, et al., Cancer Res 59:102 (1999). There is also evidence that KRN700-stimulated NKT cells can induce NK cell proliferation and enhance NK cell killing of tumor cell lines in vitro and tumors in vivo. Thus, Vα24⁺Vβ11⁺ NKT cells can be directly or indirectly responsible for tumor cell eradication.

Alterations in Vα24⁺ T cells have been seen in patients with a wide variety of diseases. Reductions in human Vα24⁺ T cell numbers and alterations in cytokine secretion have been linked to progression of or tissue damage associated with human autoimmune disorders (Tahir, et al., J Immunol 167:4046 (2001); Gumperz, et al., J Exp Med 195:625 (2002); Kita, et al., Gastroenterology 123:1031 (2002); Sumida, et al., J Exp Med 182:1163 (1995); Oishi, et al., J Rheumatol 28:275 (2001)), prostate cancer (Van Der Vliet, et al., Immunology 98:557 (1999)), and primary biliary cirrhosis (Gumperz, et al., J Exp Med 195:625 (2002)). In contrast, increases in Vα24⁺ T cells have been seen in patients with atopy (Van Der Vliet, et al., Clin Immunol 100:144 (2001)), chronic viral hepatitis (Magnan, et al., Allergy 55:286 (2000)), and myasthenia gravis Nuti, et al., Eur J Immunol 28:3448 (1998). The role of Vα24⁺ T cells or T cells which respond to KRN7000 in the treatment of autoimmune diseases, infectious disease, and cancer is supported by disease models in mice (reviewed in Reinhardt and Melms Neurology 52:1485 (1999); Kronenberg and Gapin, Nat. Rev. Immunol. 2:557 (2002)) which show that overexpression of these T cells or in vivo treatment with KRN7000 can prevent or cure colitis (Hammond and Godfrey, Tissue Antigens 59:353 (2002)), tumor metastases, diabetes, and enhance the potency of vaccines (Saubermann, et al., Gastroenterology 119:119 (2000)).

In subjects who show altered function, decreased, or no detectable Vα24⁺ T cells, expansion and activation of these cells either in vivo or in vitro with subsequent adoptive transfer may be therapeutically beneficial, especially for selective killing of malignant cells or for treatment of some autoimmune diseases. The invention addresses this need and provides related benefits.

### SUMMARY

The invention provides methods for stimulating the proliferation of human T cells in vitro. In one embodiment, a method includes repeatedly culturing donor T cells in the presence of antigen presenting cells, an antigen, a cell survival factor and serum for at least 7 days under conditions stimulating proliferation of the T cells. In another embodiment, a method includes repeatedly culturing donor T cells in the presence of antigen presenting cells, an antigen, IL-2, without IL-7 or IL-15, and serum under conditions stimulating proliferation of the T cells. In various aspects, proliferated T cells express Vα24 T cell receptor (TCR), CD3 or CD161; are capable of killing a cell; produce one or more cytokines; exhibit anti-tumor cell activity; activate NK cells to exhibit anti-tumor cell activity. In additional aspects, antigen presenting cells are human or are non-human; are from the same human as the human donor T cells or are from a different human; are present in or obtained from human peripheral blood mononuclear cells (PBMC); are viable or non-viable (e.g., irradiated); are dendritic, B-, monocyte or macrophage cells; are engineered to express human or non-human CD1a, CD1b, CD1c or CD1d, or a molecule having the glycolipid binding activity of human or non-human CD1a, CD1b, CD1c or CD1d. In another aspect, cells are passaged at least five times.

Serum useful in accordance with the invention include, for example, human or non-human serum, or a serum-free substitute medium. Cell survival factors useful in accordance with the invention include, for example, molecules that bind to a molecule on the surface of a T cell; a cytokine; or an interleukin (IL-2, IL-7 or Il-15) or interferon. Antigen useful in accordance with the invention include, for example, glycosphingolipid (e.g., KRN 7000 or a KRN 7000 analogue such as β-glucosylceramide), a bacterial antigen (e.g., tetanus toxoid, diphtheria toxin, BCG, pertussis antigen, Hemophilus influenzae type B antigen or a pneumoccoccol antigen) or a viral antigen (e.g., measles virus antigen, rubella viral antigen, varicella viral antigen, or hepatitis viral antigen). Donor T cells may be non-sensitized or sensitized with one or more antigens.

Methods of the invention include cells proliferating to about 10⁸ cells or greater over 10 weeks.

The invention also provides proliferated T cell cultures produced in accordance with the methods of the invention. In one embodiment, proliferated T cells comprise a cell population at least a portion of which express Vα24 or Vβ11.

The invention further provides kits including proliferated T cells. In one embodiment, a kit includes proliferated T cells comprise a cell population at least a portion of which express Vα24 or Vβ11. In another embodiment, proliferated T cells are included in a pharmaceutical formulation in the kit.

The invention further provides methods for providing cell therapy to a subject. In one embodiment, a method includes administering to the subject a T cell culture prepared in accordance with the invention in an amount sufficient to provide therapy to the subject. In one aspect, the donor T cells used for proliferation are obtained from the subject to which the proliferated T cells are administered. In additional aspects, the subject has or is at risk of having undesirable numbers of T cells (e.g., that express Vα4 T cell receptor); is a candidate for or has undergone organ or tissue transplant; has or is at risk of having an immune deficiency (e.g., associated with an organ or tissue transplant), an autoimmune disorder (e.g., diabetes, multiple sclerosis, systemic sclerosis, colitis, hepatitis, lupus, rheumatoid arthritis or Sjögren's syndrome), a cancer, or an infectious disease. In more specific aspects, the cancer comprises a solid tumor, metastatic tumor, leukemia (e.g., T cell, B cell or monocytic leukemia), lymphoma (e.g., Hodgkin's lymphoma or non-Hodgkin's lymphoma), or myeloma; an adenocarcinoma, plasmacytoma, sarcoma, carcinoma or neuroblastoma. In more specific aspects, the infectious disease is caused by a virus (e.g., human immunodeficiency virus (HIV), hepatitis C virus (HCV), cytomegalovirus or hepatitis B virus (HBV)), bacterium (e.g., causes tuberculosis, lyme disease or leprosy), fungus, or a parasite (e.g., causes malaria or Chagas' disease).

### DESCRIPTION OF DRAWINGS

FIG. 1 shows T cell and NK cell subsets in healthy donor PBMC. (top panel) Left histogram shows forward (FSC-H) and side scatter (SSC-H) of representative donor PBMC (a box is drawn around the live cells). Right histogram shows the percentage of CD3⁺ and CD161⁺ cells on live-gated cells. Bottom panel shows the percentage of T cells (CD3⁺CD161−), NKT cells (CD3⁺CD161⁺), Vα24⁺Vβ11⁺, and NK cells (CD3⁺CD161⁺) in donor PBMC.

FIG. 2 shows correlation of Vα24⁺Vβ11⁺ cells with hCD1d-KRN7000-tetramer⁺CD3⁺ cells in fresh and cultured healthy donor PBMC. (top panel) Percentage of Vα24⁺Vβ11β+ and hCD1d-KRN7000-tetramer⁺CD3⁺ cells on lymphocyte-gated PBMC was determined by flow cytometry on gated live lymphocytes (R2). (bottom panel) The frequency of Vα24⁺Vβ11⁺ and hCD1d-KRN7000-tetramer⁺ cells in culture was determined after stimulation for 7 days with KRN7000 (100 ng/ml) and IL-2 (10 ng/ml).

FIG. 3 shows that expanded and sorted Vα24⁺ T cells are functional and produce cytokines. (A) Cells sorted into Vα24⁺CD4⁺ and Vα24⁺CD4− subsets. (B) Intracellular staining of Vα24⁺ cells with antibodies to IL-2, IFN-γ, and IL-4. Histograms show staining of Vα24⁺CD4⁺ and Vα24⁺CD4− live cells. Heavy line represents staining of PMA and ionomycin-activated cells. Thin line represents staining of unstimulated cells. Numbers in histograms represent percent positive staining over unstimulated cells.

FIG. 4 shows that sorted and expanded Vα24⁺ T cells selectively kill target cells pulsed with KRN7000. The percentage of specific ⁵¹Cr release was calculated as a measure of killing. Upper panel Vα24⁺ CD4⁺ T cells, lower panel Vα24⁺CD4⁻ T cells.

### DESCRIPTION

The invention is based, at least in part, on the finding that T cells can be stimulated to proliferate in vitro by repeatedly culturing donor T cells in the presence of antigen presenting cells, an antigen, a cell survival factor and serum. In particular, Vα24⁺Vβ11⁺ T cells were expanded in vitro to large numbers of cells by repeated stimulation with autologous donor PBMCs, KRN7000, and IL-2. After only 5 weeks of repeated culturing, a more than one million-fold expansion of Vα24⁺Vβ11⁺ cells was achieved. Additional repeated culturing with allogeneic PBMCs, KRN7000, and IL-2 for 10 weeks, produced up to 10⁹ Vα24⁺ T cells. After sorting with Vα24 antibody, T cell lines could be passaged at least 11-times and could be expanded to greater than 10⁹ cells over 10 weeks. Expanded CD4⁺, CD8⁺, and CD4⁻8⁻ (CD4⁻) subsets of Vα24⁺Vβ11⁺ T cells were functional. For example, the expanded cells secreted cytokines and exhibited cytotoxic activity (e.g., killed tumor cells in the presence of KRN7000, and activated NK cells to kill tumor cell lines in vivo).

The invention expanded T cells may be used for treatment of various T cell associated conditions or disorders. For example, conditions or disorders characterized by immune deficiency, autoimmunity, undesirable immune response such as graft vs. host or allergy, hyperproliferative and disorders such as cancers and autoimmune diseases may be treated with the expanded T cells.

The invention therefore provides methods of expanding T cells, producing populations of expanded T cells, and methods of treating subjects employing the expanded T cell populations. In one embodiment, a method includes repeatedly culturing donor T cells in the presence of antigen presenting cells, an antigen, a cell survival factor and serum for at least 7 days under conditions stimulating proliferation of the T cells. In one aspect, at least a portion of the proliferated T cells express Vα24 T cell receptor (TCR). In another aspect, at least a portion of the proliferated T cells express CD3 or CD161. In yet another aspect, at least a portion of the proliferated T cells are capable of killing a cell or activating other cells to kill a cell.

As used herein, the phrases “repeatedly cultured” or “repeated stimulation” and grammatical variations thereof, means that the cells are grown under conditions allowing their survival or proliferation, typically for one or more passages. Proliferated cultured cells grown until they reach an appropriate cell density (e.g., 10⁵ to 10⁹ cells/ml, typically approximately 2×10⁶ cells/ml). The cells are passaged or split to lower cell density (e.g., diluted 1:10, 1:5, 1:4, 1:3, 1:2, etc.) into fresh medium, antigen presenting cells, cell survival factor, etc., if needed, and the T cells continue to proliferate, thereby expanding the T cell population. In this way, large quantities of T cells may be produced by repeatedly culturing or passaging proliferating T cells.

As used herein, an “antigen presenting cell” means any cell capable of presenting an antigen for the purpose of stimulating or inhibiting (e.g., tolerizing) an immune response to the antigen. Antigen presenting cells may be from the same human as the human donor T cells are from may be from a different human. Antigen presenting cells may also be from a non-human, e.g., a primate. Antigen presenting cells may be viable or non-viable. For example, antigen presenting cells may be treated to be inviable before practicing a method of the invention. in one embodiment, antigen presenting cells are irradiated. Antigen presenting cells may also be engineered to express a protein or manipulated genetically or otherwise. For example, in one particular embodiment, antigen presenting cells are engineered to express human or non-human CD1a, CD1b, CD1c, CD1d or a molecule having the glycolipid binding activity of human or non-human CD1a, CD1b, CD1c, CD1d.

Antigen presenting cells are present and, therefore, may be obtained from peripheral blood mononuclear cells (PBMC). Thus, in another embodiment, antigen presenting cells comprise human (autologous or non-autologous) peripheral blood mononuclear cells Particular non-limiting examples of antigen presenting cells include dendritic cells, B cells, macrophages and monocytes. Antigen presenting cells further include progenitors of such cells, even though the progenitor cells may be incapable of presenting antigen. Antigen presenting cells may optionally be added to the repeatedly cultured T cells at any time point in order to restimulate the T cells to proliferate.

Equivalents of antigen presenting cells useful in accordance with the invention include molecules such as antibodies and ligands (e.g., genetically engineered physiological ligand) that binds T cell receptor complex, which consists of CD3 chains (gamma, delta, epsilon and zeta) and T cell receptor chains (alpha and beta). Exemplary monoclonal antibodies include, for example, anti-Vα24, anti-Vβ11 (Beckman-Coulter), anti-CD3. Exemplary ligand include, for example, genetically engineered CD1d molecule (human or mouse monomer, dimer or tetramer) loaded with glycolipid (e.g., KRN7000 or an analogue). Additional equivalents of antigen presenting cells useful in accordance with the invention include phorbol ester (e.g., PMA) and a calcium ionophore (e.g., ionomycin). In addition to antigen presenting cells and equivalents thereof described herein and known in the art, ligands including antibodies to marker molecules such as CD28, CD134, CD137, CD11a, CD54 and CD2 may be added to the cells to augment proliferation or survival. Ligands to the aforementioned protein markers are commercially available.(e.g., soluble OX40-ligand that binds OX40 and CD137-ligand are available from Alexis Biochemicals). Antibodies to the aforementioned markers are also commercially available. Thus, T cells can be stimulated or induced by such molecules in addition to antigen presenting cells or their equivalents in the invention methods.

Antigen presenting cells may therefore be replaced with or supplemented with the aforementioned ligands, T cell receptor binding molecules and antibodies. For example, following sorting of Vα24Vβ11 cells, antibody to CD3, Vα24, Vβ11, etc. may be used with loaded KRN7000 (e.g., CD1d tetramer loaded KRN7000) to expand the T cells without antigen presenting cells.

The term “ligand” means any molecule that binds to the reference entity, e.g. a CD marker. Such ligands include molecules that are specific that is, they preferentially bind to the entity. However, ligands also include molecules that may not specifically bind to the entity. For example, a ligand of CD28 can bind to CD28 but also may bind to one or more different molecules that are unrelated to CD28 or are related to CD28 by sequence or structure.

Donor T cells may be obtained from mammalian subjects, such as humans. Donor T cells may comprise a mixture of cells, such as PBMCs, of which the T cells in the mixture may comprise a very small percentage (e.g., 0.01-0.1% of the cells) or a very large percentage (e.g., 20-50% or more of the cells) of the total number of cells in the mixture.

Donor T cells may be antigen sensitized. For example, donor T cells may be obtained from a subject that has been previously exposed to an antigen. Particular antigens include, for example, bacterial antigen, such as tetanus toxoid, diphtheria toxin, BCG, pertussis antigen, Hemophilus influenzae type B antigen and pneumoccoccol antigen; viral antigens, such as measles virus, rubella virus, varicella virus, or hepatitis virus antigens; fungi, such as yeast, a parasite, such as malaria.

Donor cells that proliferate produce progeny cells. Donor cells and progeny thereof may be cultured or repeatedly cultured or passaged for any period of time, typically from 1 to 3 or 1 to 5 days or longer, e.g., from 5 to 7, 5 to 10, 5 to 14 days. The cells may optionally be cultured, and the cell cultures repeatedly cultured or passaged for longer periods of time, for example, 7 to 10, 7 to 14, 10 to 14, 10 to 21, 14 to 28, 14 to 35, 14 to 42 days or longer.

Donor cells and progeny thereof may be stimulated multiple times with antigen presenting cells (e.g., 1-, 2-, 3-, 4-, 5-, times or more at one or more passages). Thus, following the initial culture with antigen presenting cells, antigen, cell survival factor and serum, additional antigen presenting cells or equivalents, can be added one or more times to the cultured or repeatedly cultured or passaged T cells at any time. Donor cells and progeny thereof may also be stimulated multiple times with antigen and/or cell survival factor (e.g., 1-, 2-, 3-, 4-, 5-, times or more at one or more passages). Again, additional antigen or cell survival factor may be added at any time to the initial cultured or repeatedly cultured or passaged T cells.

In various embodiments, particular T cell subsets can be expanded within the culture. This may be achieved by preferential proliferation of the particular T cell subsets, or, alternatively, by sorting particular T cell subsets and reculturing them to proliferate. Thus, in various embodiments, T cells having particular characteristics, that express a particular T cell receptor (e.g., Vα24Vβ11), produce one or more cytokines or particular types of cytokines, or that have particular activities, such as direct or indirect cell killing activity (e.g., activate NK cells to exhibit anti-tumor cell activity), can be preferentially stimulated to proliferate. For example, in one aspect, Vα24⁺Vβ11⁺ cells are preferentially expanded, with or without cell sorting. In another aspect, T cells having direct or indirect cell killing activity are preferentially expanded.

As used herein, “cell survival factor” refers to a molecule that stimulates cell survival, proliferation, differentiation, or that modulates cell motility or that inhibits or prevents cell death (e.g., apoptosis or programmed cell death). Particular non-limiting examples of such factors include cytokines, such as interleukins (e.g., IL-2, IL-7 and IL-15) and interferons, chemokines, anti-apoptotic factors, antigens, or cell determinants or markers typically located on the surface of cells of the immune system, referred to as “CD” molecules. T cell surface molecules that function as cell survival factors include, for example, CD28, CD 134 (also known as OX40), CD137 (also known as 4-1BB), CD11a (also known as LFA-1), CD54 (also known as ICAM-1), and CD2. Antibodies, ligands or engineered ligands which bind to the T cell surface molecule also function as cell survival factors. Particular examples include CD28 ligands such as CD80 and CD86; CD134 ligand such as OX40L (OX40 ligand); CD137 ligand such as 4-1BBL (4-1BB ligand); CD11a ligands such as ICAM-1, ICAM-2, and ICAM-3; and CD54 ligand such as CD11a.

Cell survival factors may be added as a single stimulation dose. Cell survival factors may optionally be added to the repeatedly cultured T cells multiple times, at the same or varying concentration, in order to extend or prolong T cell survival. For example, a cell survival factor may be added to the repeatedly cultured T cells every 7 to 9 days.

Antigens include glycosphingolipids. Exemplary glycosphingolipid antigens are KRN7000 (alpha-galactosylceramide,(2S,3S,4R)-1-O-(alpha-D-Galactopyranosyl)-N-hexacosanoyl-2-amino-1,3,4-octadecanetriol, Kirin Brewery, Co., Ltd., Tokyo, Japan), and KRN7000 analogues. As used herein, the term “KRN7000 analog” means a molecule that functions in the way KRN7000 does, e.g., binds CD1d and activates Vα24⁺Vβ11⁺ cells. Thus, such molecules include KRN7000 derivatives as well as compounds whose structure is similar to KRN7000. One particular example of a KRN7000 analog is β-glucosylceramide (β-GluCer). Other KRN7000 analogues are known in the art and are applicable in the invention, for example, analogues described in WO 93/05055; WO 94/09020; WO 94/24142; WO 94/02168; JP5009193A2; and U.S. Pat. No. 2002/0,032,158.

Antigens also include bacterial antigens. Particular non-limiting examples include tetanus toxoid, diphtheria toxin, BCG, pertussis, Hemophilus influenzae type B and pneumoccoccol antigens. Additional examples include viral antigens. Particular non-limiting examples include measles, rubella, varicella and hepatitis viral antigens.

Serum from any mammal is applicable in the invention. Serum can be obtained from a human subject that from which donor T cells were obtained, or from a different human subject, or from a non-human. Serum equivalents or serum-free medium may be substituted for mammalian serum. Such serum equivalents and serum-free medium containing factors such as albumin and growth factors enabling cell survival and proliferation are known in the art and, additionally, are commercially available (e.g., AIM V, Gibco-BRL, Inc.). It is likely that human serum will provide the greatest T cell proliferative capacity.

As used herein, the phrases “without a cell survival factor” or “in the absence of a cell survival factor,” for example, when used in reference to a cytokine such as IL-7 or IL-15, means that exogenous IL-7 or IL-15 is not added to the cells during culturing or repeated culturing of donor cell progeny. However, there may be small amounts of cell survival factor (e.g., IL-7 or IL-15) present in the donor T cells or antigen presenting cells. For example, small amounts of a cell survival factor may be present in donor cells or in peripheral blood mononuclear cell due to the cells being isolated from their natural in vivo environment. The phrases therefore do not exclude situations where small amounts of one or more cell survival factors are present in the cultured T cells.

As used herein, the term “bind” and grammatical variations thereof means that the compositions referred to have affinity for each other. “Specific binding” is where the binding is selective between two molecules. A particular example of specific binding is that which occurs between ligand and a receptor T cell surface molecule. Binding affinity is typically represented quantitatively by the dissociation constant (K_{D}) between the two molecules. Typically, specific binding is distinguished from non-specific binding when the dissociation constant (K_{D}) is less than about 1×10⁻⁵ M or less than about 1×10⁻⁶ M or 1×10⁻⁷ M or 1×10−8. Specific binding can be detected, for example, by ELISA, immunoprecipitation, coprecipitation, with or without chemical crosslinking, two-hybrid assays and the like.

The term “antibody” refers to a protein that binds to other molecules (antigens) via heavy and light chain variable domains, V_{H} and V_{L}, respectively. Antibodies include polyclonal or monoclonal IgG, IgD, IgA, IgM and IgE. Antibodies may be intact immunoglobulin molecules, two full length heavy chains linked by disulfide bonds to two full length light chains, as well as subsequences (i.e. fragments) of immunoglobulin s, with our without constant region, that bind to an epitope of an antigen, or subsequences thereof (i.e. fragments). Specific examples of antibody subsequences include, for example, Fab, Fab′, (Fab′)₂, Fv, or single chain antibody (SCA) fragment (e.g., scFv).

Antibodies contain kappa or lambda chain sequences. Full length antibody contains two kappa or two lambda light chains. The primary difference between kappa and lambda light chains is in the sequences of the constant region. In humans, the kappa chain variable region sequences have more diversity than lambda chain variable region sequences which results in the generation of more different (diverse) antibodies.

Ligands also include modified forms such as sequences having one or more amino acid substitutions (e.g., conservative substitutions, or a human or primate amino acid substituted for a non-human or nonprimate amino acid), additions or deletions, provided the modification does not destroy function. For example, a modified antibody retains, at least in part, antigen binding activity. The term “modify” and grammatical variations thereof therefore denotes an alteration of a molecule that does not destroy all activity of the modified molecule.

Modifications also include derivatized sequences, for example, amino acids in which free amino groups form amine hydrochlorides, p-toluene sulfonyl groups, cabrobenzoxy groups; the free carboxy groups from salts, methyl and ethyl esters; free hydroxl groups that form O-acyl or O-alkyl derivatives, as well as naturally occurring amino acid derivatives, for example, 4-hydroxyproline, for proline, 5-hydroxylysine for lysine, homoserine for serine, ornithine for lysine, etc. Also included are modifications that confer covalent bonding, for example, a disulfide linkage between two cysteine residues thereby producing a cyclic polypeptide. Modifications also include addition of functional entities such as tags (e.g., polyhistidine, T7, immunoglobulin, etc.), gold particles, covalently or non-covalently attached to antibody. Modifications further include radioactive or alternatively non-radioactive detectable labels attached to or incorporated into the molecule. Modifications can be produced using any of a variety of methods well known in the art (e.g., PCR based sited-directed, deletion and insertion mutagenesis, chemical modification and mutagenesis, cross-linking, etc.).

As used herein, the term “subsequence” or “fragment” means a portion of the full length molecule. Thus, a subsequence of an antibody is one or more amino acids less in length than full length polypeptide (e.g. one or more internal or amino or carboxy-terminal amino acid deletions). Subsequences therefore can be any length up to one amino acid less than the full length molecule.

The preparation of polyclonal antibodies and their purification is well known in the art (see, e.g., Green et al. (1992) In: Immunochemical Protocols, pages 1-5, Manson, ed., Humana Press; Harlow et al. (1988), supra; and Coligan et al. (1994) In: Current Protocols in Immunolog, Wiley; and Barnes et al. (1992) In: Methods in Molecular Biology, Vol. 10, pages 79-104, Humana Press). Alternatively, spleen cells from animals that express antibody may be fused with a myeloma cell to produce a hybridoma thereby producing monoclonal antibody. The selected secreting hybridomas are then cultured either in vitro (e.g., in tissue culture), or in vivo (as ascites in mice) and antibodies purified. Antibodies may also be isolated or purified by other techniques well known in the art (see Antibodies: A Laboratory Manual, Harlow and Lane (eds.), Cold Spring Harbor Laboratory Press, 1988). Monoclonal antibodies may also be generated using other techniques (see, e.g., U.S. Pat. Nos. 4,902,614, 4,543,439, and 4,411,993; see also Monoclonal Antibodies, Hybridomas: A New Dimension in Biological Analyses, Plenum Press, Kennett, McKearn, and Bechtol (eds.), 1980).

Proliferated T cells of the invention can be introduced into a subject, the same subject that provided the donor cells, or a different subject, thereby increasing numbers of T cells in the subject. The method thus provides cell therapy to the subject. The invention therefore also provides methods of treating a T cell associated disorder or condition.

In one embodiment, a method includes administering to the subject a T cell culture produced in accordance with the invention sufficient to provide therapy to the subject. In one aspect, the donor T cells are obtained from the subject to which the proliferated T cells are administered. In another aspect, the subject has or is at risk of having undesirable numbers of T cells. In additional aspects, the subject has or is at risk of having undesirable numbers of T cells that express Vα24 T cell receptor; the subject is a candidate for or has undergone organ or tissue transplant; the subject has or is at risk of having an immune deficiency (e.g., associated with an organ or tissue transplant), an autoimmune disorder, a cancer, or an infectious disease.

As used herein, the term “T cell associated disorder” means any undesirable physiological condition or pathological disorder in which increasing numbers of T cells or providing T cells having one or more particular beneficial T cell activities, such as cell killing activity, the ability to produce one or more cytokines, or the ability to stimulate beneficial activities in other immune cells, may ameliorate one or more undesirable symptoms of the condition or disorder, or reduce one or more causes of the condition or disorder. Thus, a T cell associated condition or disorder need not be caused by abnormalities or deficiencies in T cell numbers or T cell activities and, therefore, may be completely unrelated to the T cells in the subject. Rather, a physiological condition or pathological disorder need only be treatable with or respond to the proliferated T cells of the invention in order to be considered a T cell condition or disorder, as used herein. For example, introducing proliferated T cells having anti-tumor activity into a subject having a tumor may reduce the size of the tumor or prevent further increases in tumor size or metastasis, thereby providing a therapeutic benefit to the subject, even though the subject's T cells are not abnormal or deficient.

Target subjects therefore include those having a T cell associated condition or disorder as described herein or known in the art as well as subjects amendable to treatment with a population of proliferated T cells of the invention.

The term “subject” refers to animals, typically mammalian animals, such as a non human primate (apes, gibbons, chimpanzees, orangutans, macaques), a domestic animal (dogs and cats), a farm animal (horses, cows, goats, sheep, pigs), experimental animal (mouse, rat, rabbit, guinea pig) and humans. Subjects include animal disease models (e.g., immune deficient or autoimmune mice).

Particular examples of subjects that may be treated in accordance with the invention include subjects having or at risk of having graft vs. host disease, inflammation or an undesirable immune response, autoimmune disease, immune-deficiency and cell proliferative (e.g., hyperproliferative) and/or differentiative disorders. As used herein, the term “proliferative disorder” means a pathological or non-pathological physiological condition characterized by aberrant cell proliferation or cell survival (e.g., due to deficient apoptosis). The term “differentiative disorder” means a pathological or non-pathological physiological condition characterized by aberrant or deficient cell differentiation.

Subjects also include, for example, those having or at risk of having a pathogen infection (e.g., viral, such as human immunodeficiency virus (HIV), hepatitis C virus (HCV), cytomegalovirus and hepatitis B virus (HBV); bacterial such as tuberculosis, lyme disease or leprosy; fungal, such as yeast; parasitic such as malaria (plasmodium) or Chagas' disease (Trypanosoma cruzi), mycoplasmsa, etc.); those receiving a vaccine (e.g., against virus, bacteria, fungi, parasite, mycoplasmsa, etc.); those having or at risk of having a hyperproliferative condition (e.g., a metastatic or non-metastatic cancer or tumor); and those having or at risk of having an autoimmune disorder or disease.

Autoimmune diseases include, for example, diabetes mellitus, arthritis (including rheumatoid arthritis, juvenile rheumatoid arthritis, osteoarthritis, psoriatic arthritis), multiple sclerosis, encephalomyelitis, myasthenia gravis, systemic lupus erythematosis, autoimmune thyroiditis, dermatitis (including atopic dermatitis and eczematous dermatitis), psoriasis, Sjogren's Syndrome, Crohn's disease, aphthous ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic sprue, lichen planus, Graves' disease, sarcoidosis, primary biliary cirrhosis, uveitis posterior, production of auto-antibodies, and interstitial lung fibrosis

Undesirable immune responses include, for example, inflammation or an allergic reaction to antigen or antibiotics, such as atopic allergy; vascular inflammatory disease such as artherosclerotic lesions, plaque disruption and thrombus formation; and subjects which have or are going to have a cell, tissue, or organ transplant (e.g., graft v. host disease). Particular non-limiting examples of transplants in which graft vs. host disease may arise include bone marrow, blood vessels, kidney, liver, heart, lung, pancreas and skin. Transplantation includes grafting of tissues or organ from the body of an individual to a different place within the same or a different individual. Transplantation of tissues or organs between genetically dissimilar animals of the same species is termed as allogeneic transplantation. Transplantation of animal organs into humnans is termed xenotransplants.

Proliferative or differentiative disorders or conditions amenable to treatment include diseases and physiological conditions, both benign and neoplastic, characterized by abnormal or undesirable cell numbers, cell growth or cell survival in a subject. The term is meant to include all types of cancerous growths or oncogenic processes, metastatic tissues or malignantly transformed cells, tissues, or organs, irrespective of histopathologic type or stage of invasiveness.

Examples of cellular proliferative and/or differentiative disorders include cancer, e.g., carcinoma, sarcoma, adenocarcinoma, neuroblastoma, metastatic disorders or hematopoietic neoplastic disorders, e.g., lymphomas, leukemias, plasmacytomas and myelomas.

A metastatic tumor can arise from a multitude of primary tumor types, including but not limited to those of breast, lung, thyroid, larynx, head and neck, brain, lymphoid, gastrointestinal (mouth, esophagus, stomach, small intestine, colon, rectum), genito-urinary tract (uterus, ovary, cervix, bladder, testicle, prostate), kidney, pancreas, liver, bladder, bone, muscle, skin, etc.

Carcinomas refer to malignancies of epithelial or endocrine tissue, and include respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from the cervix, lung, prostate, breast, head and neck, colon, liver and ovary. The term also includes carcinosarcomas, e.g., which include malignant tumors composed of carcinomatous and sarcomatous tissues. Adenocarcinoma includes a carcinoma of a glandular tissue, or in which the tumor forms a gland like structure.

Sarcomas refer to malignant tumors of mesenchymal cell origin. Exemplary sarcomas include for example, lymphosarcoma, liposarcoma, osteosarcoma, and fibrosarcoma.

Additional examples of proliferative disorders include hematopoietic neoplastic disorders. As used herein, the term “hematopoietic neoplastic disorders” includes diseases involving hyperplastic/neoplastic cells of hematopoietic origin, e.g., arising from myeloid, lymphoid or erythroid lineages, or precursor cells thereof. Typically, the diseases arise from poorly differentiated acute leukemias, e.g., erythroblastic leukemia and acute megakaryoblastic leukemia. Additional exemplary myeloid disorders include, but are not limited to, acute promyeloid leukemia (APML), acute myelogenous leukemia (AML) and chronic myelogenous leukemia (CML) (Vaickus, L. Crit. Rev. in Oncol./Hemotol. 11:267(1991)). Lymphoid malignancies include, but are not limited to acute lymphoblastic leukemia (ALL) which includes B-lineage ALL and T-lineage ALL, chronic lymphocytic leukemia (CLL), prolymphocytic leukemia (PLL), hairy cell leukemia (HLL) and Waldenstrom's macroglobulinemia (WM).

Additional forms of malignant lymphomas include, but are not limited to Hodgkin lymphoma, non-Hodgkin lymphoma and variants thereof, peripheral T cell lymphomas, adult T cell leukemiallymphoma (ATL), cutaneous T-cell lymphoma (CTCL), large granular lymphocytic leukemia (LGF) and Reed-Stemberg disease.

Target subjects also include those at risk of developing a T cell associated condition or disorder, for example, a subject at risk of developing a tumor (e.g., identified through genetic screening such as women at risk for breast cancer due to mutations in brea or inheritance, a biopsy such as a colonoscopy in which benign polyps in the colon indicate a propensity to develop colon cancer, a test of a biological fluid, such as urine in men in which elevated levels of prostate specific antigen indicate increased risk for prostate cancer, etc.). The invention methods are therefore applicable to treating a subject who is at risk of developing a T cell associated condition or disorder, but who has not yet exhibited overt symptoms of the condition or disorder.

T cells can be administered prophylactically to a subject prior to onset of T cell associated condition or disorder. For example, a subject about to be treated with an immunosuppressing agent (e.g., a steroid) can be administered T cells in order to inhibit immunosuppression in the subject that occurs typically following treatment with the immunosuppressing therapy. Prophylactic methods are therefore also included.

At risk subjects appropriate for treatment can therefore be identified as having a genetic predisposition or family history towards developing a T cell associated condition or disorder compared to appropriate control subjects. Such subjects can be identified using routine genetic screening, inquiry into the subjects' family history to establish that they are at risk of the condition or disorder, a biopsy of a tissue or a screen of a biological fluid for the presence or absence of a molecule indicating that the subject is at increased risk of the condition or disorder.

The methods of the invention, including treating a T cell associated condition or disorder of a subject, likely results in an improvement in the subjects' condition or disorder which includes, for example, a reduction in severity or duration of one or more symptoms of the condition or disorder, or decreasing the subject's risk for developing symptoms associated with a T cell associated condition or disorder. Improvements therefore include decreasing one or more symptoms associated with immune deficiency, autoimmunity, allergy, hyperproliferation or a hyperplastic condition such as a tumor or cancer, pathogen infection, which are all satisfactory clinical endpoints.

An improvement also includes reducing the need for other therapies being used to treat the condition or disorder. For example, a reduction in the frequency or amount (dosage) of a drug used for treating a subject having or at risk of having a T cell associated condition or disorder. In particular, autoimmune patients treated with a steroid may require less steroid when treated in combination with proliferated T cells of the invention. An improvement therefore includes reducing the dosage frequency or amount of a steroid that the subject was administered in comparison to the dosage frequency or amount administered prior to treatment with a proliferated T cells of the invention.

An improvement may have a relatively short duration, e.g., the improvement may last several hours, days or weeks, or extend over a longer period of time, e.g., months or years. A treatment of the invention need not be a complete ablation of any or all symptoms of the T cell associated condition or disorder. For example, reducing severe rheumatoid arthritis to a less severe form is an improvement. Likewise, reducing the frequency or dosage of insulin in a diabetic subject is an improvement. Thus, a satisfactory clinical endpoint is achieved when there is a subjective or objective detectable improvement in the subjects' condition, for a short or long period of time.

Amounts of T cells administered, are typically in an “effective amount,” that is an amount sufficient to produce the desired affect, e.g., a therapeutic effect or an improvement in the T cell associated condition or disorder, or a symptom thereof, as set forth herein. For example, where it is desired to increase the number of T cells in a subject, the effective amount will be that which detectably increases the number of T cells. Where it is desired to treat a solid tumor in a subject, the effective amount will be that which detectably decreases the size of the tumor or inhibits or prevents further increases in tumor size of at least part of the tumor (e.g. 10% of the cells, or 20% or more), or inhibits metastasis of the tumor, all of which are satisfactory clinical endpoints. Examination of a solid tumor using invasive or non-invasive imaging methods can ascertain a reduction in tumor size, or inhibiting increases in the size of the tumor.

Of course, treating a subject in accordance with the invention further includes supplementing with other therapies that can be used to treat a T cell associated condition or disorder. Other therapies include drug treatment, surgical resection, transplantation, radiotherapy, etc. The T cells can be administered prior to, contemporaneously with or following other treatment protocols. For example, proliferated T cells may be used with an immunopotentiating drug or another therapeutic protocol (e.g., cytokine therapy, chemotherapy, surgical resection, etc.) for increasing immune responsiveness to a pathogen or to a cancer. Alternatively, proliferated T cells that may be used with an immunosuppressive drug (e.g., a steroid) or another therapeutic protocol for treating an autoimmune disorder, inflammation or for inhibiting transplant rejection as in graft vs. host disease.

Proliferated T cells can be administered to a subject as a single or multiple dose e.g., one time per week for between about 1 to 10 weeks, or for as long as appropriate, for example, to achieve a reduction in the duration or severity of one or more symptoms of a T cell associated condition or disorder. Doses can vary depending upon the particular condition or disorder being treated, the extent or severity of the condition or disorder, the clinical endpoint desired, previous or simultaneous treatments, the general health, age, sex or race of the subject and other factors that will be appreciated by the skilled artisan. Such factors may influence the dosage and timing required to provide an amount sufficient for therapeutic benefit. Doses can be empirically determined or determined using animal disease models or optionally in human clinical trials. In the methods of the invention, including prophylactic and therapeutic treatments, the methods doses or protocols may be specifically tailored or modified based on pharmacogenomic data.

The invention further provides pharmaceutical compositions. Such pharmaceutical compositions are useful for administration to a subject in vivo or ex vivo, and for treating a T cell associated condition or disorder in order to practice the methods of the invention, for example.

Pharmaceutical compositions include “pharmaceutically acceptable” and “physiologically acceptable” carriers, diluents or excipients. As used herein the term “pharmaceutically acceptable” and “physiologically acceptable” includes solvents (aqueous or non aqueous), solutions, emulsions, dispersion media, compatible with administration of proliferated T cells.

Pharmaceutical compositions can be formulated to be compatible with a particular route of administration. Thus, pharmaceutical compositions include carriers, diluents, or excipients suitable for administration by various routes. Pharmaceutical formulations and delivery systems are known in the art (see, e.g., Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms, Technonic Publishing Co., Inc., Lancaster, Pa., (1993); and Poznansky et al., Drug Delivery Systems, R. L. Juliano, ed., Oxford, N.Y. (1980), pp. 253-315)

The invention further provides kits including proliferated T cells and pharmaceutical formulations thereof, optionally packaged into suitable packaging material. A kit typically includes a label or packaging insert including a description of the components or instructions for use in vitro, in vivo, or ex vivo, of the components therein.

The term “packaging material” refers to a physical structure housing the components of the kit. The packaging material can maintain the components sterilely, and can be made of material commonly used for such purposes (e.g., paper, corrugated fiber, glass, plastic, foil, ampules, etc.). Each component of the kit can be enclosed within an individual container and all of the various containers can be within a single package. Invention kits can be designed for cold storage.

The label or packaging insert can include appropriate written instructions. Kits of the invention therefore can additionally include labels or instructions for using the kit components in any method of the invention. Instructions can include instructions for practicing any of the methods of the invention described herein including treatment methods.

The instructions may be on “printed matter,” e.g., on paper or cardboard within or affixed to the kit, or on a label affixed to the kit or packaging material, or attached to a vial or tube containing a component of the kit. Instructions may additionally be included on a computer readable medium, such as a disk (floppy diskette or hard disk), optical CD such as CD- or DVD-ROM/RAM, magnetic tape, electrical storage media such as RAM and ROM and hybrids of these such as magnetic/optical storage media.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described herein.

All publications, patents and other references, GenBank citations and ATCC citations cited herein are incorporated by reference in their entirety. In case of conflict, the specification, including definitions, will control.

As used herein, the singular forms “a”, “and,” and “the” include plural referents unless the context clearly indicates otherwise. Thus, for example, reference to “a T cell function or activity” includes a plurality of such functions or activities and reference to “a T cell” can include reference to one or more T cells and so forth.

The following abbreviations are used: PBMC, peripheral blood mononuclear cells; KRN7000, □-galactosylceramide derived from marine sponge; Vα24, alpha chain of the T cell receptor; Vβ11, beta chain of the T cell receptor; NK, natural killer cell; NKT, natural killer T cell; TT, tetanus toxoid; rhIL-2, recombinant human interleukin-2; hCD1d-KRN7000-tetramer, human CD1d-tetramer loaded with KRN7000; and FCS, fetal calf serum.

A number of embodiments of the invention have been described. Nevertheless, it will be understood that various modifications may be made without departing from the spirit and scope of the invention. Accordingly, the following examples are intended to illustrate but not limit the scope of invention described in the claims.

### EXAMPLES

### Example 1

This example describes materials and methods.

Cell Isolation

Heparinized venous blood was obtained from healthy adult volunteers at Scripps Research Institute, La Jolla, Calif. (GCRC normal blood drawing program). Peripheral blood mononuclear cells (PBMC) were obtained by density-gradient centrifugation using Histopaque-1077 (Sigma Diagonistics, Inc., St. Louis, Mo.). Cells were frozen at 10-20×10⁶/ml in medium containing 90% fetal calf serum (catalog # 26300-061, GibcoBRL, Grand Island N.Y.) or 90% pooled human serum (ICN Biomedicals, Inc, Aurora, Ohio), and 10% DMSO (catalog #: BP231-1, Fisher Scientific, Fair Lawn, N.J.), placed in cryovials (catalog #: 5000-0012, Nalge Co., Rochester, N.Y.) and stored in liquid nitrogen.

Antigens

KRN7000 (α-galactosylceramide, Kirin Brewery Co., Ltd., Tokyo, Japan), a glycosphingolipid derived from the marine sponge Agelas mauritanius, was used to stimulate PBMCs. KRN7000 analog β-galactosylceramide (β-GalCer) was also obtained from Kirin. Stock solutions were 100 μg/ml in DMSO and used at a final concentration of 100 ng/ml. Vehicle control cultures contained 0.1% DMSO. Tetanus toxoid (#8051) was obtained from the Dutch National Institutes of Health (Rijksinstituut voor Volksgezondheid en Milieuhygiene, Bilthoven, The Netherlands).

Antibodies and reagents. Anti-human Vα24-FITC and anti-human Vβ11-PE were purchased from Beckman Coulter (Miami, Fla.). Anti-human CD3-FITC and anti-human CD4-PE were purchased from BD/PharMingen (San Diego, Calif.). Human CD1d-tetramer loaded with KRN7000 (hCD1d-KRN7000-tetramer) and attached to either pycoerythrin-streptavidin (BD/PharMingen) or cychrome-streptavidin (BD/PharMingen) was made as described (Kroft and Swain, J Immunol 154:4269 (1995)) with modifications and was obtained from M. Kronenberg (La Jolla Institute for Allergy and Immunology) or A. Matsumoto (Gemini Science, Inc., San Diego, Calif.). RhIL-15 (catalog #200-15, PeproTech Inc., Rocky Hill, N.J.) for PBMC cultures was used at 100 ng/ml. Mouse anti-human CD1d antibody (42.1) (Exley, et al., Immunology 100:37 (2000)) was a gift from Mark Exley (Beth Israel Deaconess Medical Center, Boston, Mass.). Isotype control mouse IgGI was purchased from Southern Biotech (Birmingham, Ala.). FITC-labeled goat anti-mouse IgG was purchased from Jackson ImmunoResearch Laboratories, Inc. (West Grove, Pa.). Flow cytometry was performed as described (Rogers and Croft, J Immunol 163:1205 (1999)) and data was analyzed using Cellquest software.

Cell Culture

PBMCs were cultured in RPMI-1640 medium (catalog #: 11875-093, GibcoBRL) containing L-glutamine, 100 U/ml penicillin G, 100 μg/ml steptomycin sulfate (catalog #: 17-602E, BioWhittaker, Walkersville, Md.), 10 mM Hepes (catalog #: 15630-080, GibcoBRL) and 5.5×10⁻⁵ M 2-mercaptoethanol (catalog #: 21985-023, GibcoBRL). Cultures contained 10% pooled normal human serum (catalog #: 82320, ICN Biomedicals, Inc.), 10% human AB serum (catalog #: 82318, ICN), or 10% autologous donor serum. Serum was heat inactivated at 56° C. for 20 minutes before use.

For initial stimulation, PBMC were cultured at 1×10⁶ cells/ml in 48- or 24-well plates (Falcon # 3078 or 3047, Becton Dickinson and Co., Franklin Lakes, N.J.) for 7-12 days in a humidified incubator with 6% CO₂. KRN7000 was added at 100 ng/ml with 10 ng/ml rhIL-2 (catalog #: 200-02, PeproTech Inc. Rocky Hill, NJ or catalog #:23-6019, Hoffmann-La Roche, Inc., Nutley, N.J.) at the start of culture.

Restimulation Cultures

PBMC for restimulation cultures were isolated from fresh blood or prepared from vials of frozen PBMC. Cells were thawed and washed twice in RPMI. Approximately 10×106 cells were resuspend in 4 ml medium containing 10% human serum and 100 ng/ml KRN7000. Cells were incubated in 60 mm petri dishes (catalog #: 08-757-13A, Fisher Scientific, Pittsburgh, Pa.) at 37° C. for 4-5 hours. Cells were pipetted, dishes were scraped, and cells were collected into a tube and irradiated 3000 rads using ¹³⁷CS (Gammacell 40, Kanata, Canada). Debris was removed by pipetting through a nylon strainer (Falcon #2350, Becton Dickinson) and cells were washed once before use. Cell recovery was generally 50-80% of starting cell number. Unsorted NKT cells (2×10⁵/ml) were restimulated with 5 times as many autologous, KRN7000-pulsed, irradiated PBMCs in RPMI 1640 medium containing 10% human AB serum (ICN Biomedicals, Inc., Aurora, Ohio). RhIL-2 (10 ng/ml, Hoffman LaRoche, Nutley, N.J.) was given 1 day later. An aliquot of cells (generally 0.5-2×10⁶) was restimulated every 7 days. Vα24-sorted NKT cell lines were stimulated as above but contained allogeneic (instead of autologous), KRN7000-pulsed, irradiated PBMCs. No significant difference in the ability of allogeneic PBMCs to stimulate NKT cell lines was detected. Sorted Vα24⁺ T cell lines were periodically frozen, stored in liquid nitrogen, and then thawed and restimulated over a 1 year period. A total of 10-11 restimulation cycles were performed using Donor 18 (>90% CD4⁺) and Donor 20 (>95% CD4⁻) T cell lines. T cells were quantitated using trypan blue exclusion. Throughout the culture period, the sorted T cell lines remained >90% Vα24⁺ and hCD1d/KRN7000-Tetramer⁺ (Tetramer⁺). The Vα24-antibody (Beckman/Coulter) and hCD1d/KRN7000-loaded Tetramer identify the same population of T cells and were used interchangeably in these studies.

Cell Sorting

Purified Vα24⁺ cells were obtained by positive magnetic bead sorting (MACS, Miltenyi Biotec, Auburn, Calif.) using biotinylated anti-Vα24 antibody (Beckman Coulter) followed by streptavidin-labeled microbeads (Miltenyi Biotec). Alternatively, T cells were asceptically sorted into Vα24⁺CD4⁺ and Vα24⁺CD4⁺ subsets using a FACStarPIus sorter (Becton Dickinson, Mountain View, Calif.).

Cell Lines

The cell lines U937 (histiocytic lymphoma), RAJI (Burkitt's B cell lymphoma), and K562 (erythroleukemia) were originally obtained from the American Type Culture Collection, ATCC (Mannassas, Va.) and were a gift from Dr. D. Green (La Jolla Institutive for Allergy and Immunology (LIAI), San Diego, Calif.). The T cell leukemia Jurkat cell line was obtained from Nobuaki Takahashi (Kirin Brewery Co., Ltd., Gunma, Japan). Cells were maintained in RPMI-1640 with 10% fetal calf serum (GibcoBRL), penicillin, streptomycin, 2-mercaptoethanol, and Hepes as described previously. For cytotoxic assays, cells were cultured at 1×10⁵/ml with 50 ng/ml KRN7000, β-GalCer, or 0.5% DMSO (vehicle) for 16 hours prior to assay.

Chromium Release and T Cell Proliferation Assays

⁵¹Cr cytotoxicity assays were performed as follows. A total of 5×10^{3 51}Cr-labeled cells (Na₂⁵¹CrO3) (ICN Biomedicals, Inc.) from the U937, K562, or Jurkat cell line as the target cells and various numbers of effector Vα24⁺ T cells in 0.2 ml of culture medium were seeded into round-bottom microtiter wells. The culture was incubated at 37° C. in an atmosphere containing 6% CO₂ for 5 h, and 0.1 ml of supernatant was collected from each well. The percentage of specific ⁵¹Cr release was calculated as follows: [(cpm experimental−cpm spontaneous release)/(cpm maximal release−cpm spontaneous release)]×100.

Proliferation of PBMCs was measured in triplicate in 96-well flat-bottomed plates with 2×10⁵ cells/well (0.2 ml). Cells were cultured for 5 days and pulsed with [³H]-thymidine (1 μCi/well, ICN Pharmaceuticals, Irvine, Calif.) for the last 12 hours.

NK Cell Isolation and Cytotoxic Assays

NK cells were positively selected from healthy donor PBMC using a kit from Stem Cell Technologies (Vancouver, BC, Canada) (catalog #14055). Greater than 95% of cells obtained after purification were CD56⁺ CD3⁻ (data not shown). These purified NK cells (fresh NK cells) were used as effectors in a ⁵¹Cr release assay or were cultured for 3 days with supernatant from activated NKT cell lines. Alternatively, NK cells were cultured with 2 ng/ml rhIL-2 and 10 ng/ml rhIFN-γ. Supernatant from NKT cell lines was obtained after 17 hour activation on anti-Vα24-coated wells. T cells were plated at 5×10⁵/ml in a 48-well plate and incubated at 37° C. in a humidified incubator with 6% CO₂. Supernatant was collected after 17 hours, filtered through a 0.22 μM filter and added at 1:2 dilution to NK cell cultures. NK cells were plated at 1×10⁶ cells/ml in 24 or 48-well plates and cultured for 3 days prior to assay.

Cytokine Assays

Cytokine secretion was measured in supernatants by ELISA as described previously (Matsuda, et al., J Exp Med 192:741 (2000)). T cells were plated at 5×10⁵/ml in RPMI medium containing 10% human AB serum, 10 ng/ml PMA, and 500 ng/ml ionomycin. Supernatants were collected after 24 hours of activation and frozen at −80° C. Anti-IL-4 (catalog #: 14-7049), biotinylated anti-IL-4 (catalog #: 13-7048), anti-IFN-γ (catalog #: 14-7318), and biotinylated anti-IFN-γ (catalog #: 13-7319) Anti-IL10 (catalog #: 14-7108-81), biotinylated anti-IL-10 (catalog #: 13-7109-81), anti-TNFα (catalog #: 14-7348-81), biotinylated anti-TNFα (catalog #: 13-7349-81), and biotinylated anti-IL-5 (catalog #: 13-7059-81) were purchased from eBioscience (San Diego, Calif.). Anti-IL-2 (catalog #: 555051), biotinylated anti-IL-2 (catalog #: 555040), anti-GM-CSF (catalog #: 554502) and biotinylated anti-GM-CSF (catalog #: 554505) were purchased from BD PharMingen (San Diego, Calif.). Anti-IL-5 (clone TRFK5) was obtained from Michael Croft (La Jolla Institute for Allergy and Immunology, San Diego, Calif.). RhTNFα (catalog #: 300-01A), rhIL-5 (catalog #: 200-05), rhGM-CSF (catalog #: 300-03 ), and rhIL-10 (catalog #: 200-10) standards were purchased from PeproTech Inc. Limit of detection for ELISAs was 10-100 pg/ml.

Intracellular cytokine staining was performed as described (Rogers, P. R. and M. Croft, J Immunol, 163:1205 (1999)). Briefly, sorted Vα24⁺ T cells were cultured at 5×10⁵/ml in medium containing 50 ng/ml PMA, 500 ng/ml ionomycin, and 5 μg/ml Brefeldin A (Sigma, St. Louis, Mo.) for 5 hours. Cells were harvested, and stained with biotin-labeled anti-Vα24 antibody followed by cychrome-streptavidin (BD/PharMingen). Cells were fixed, permeabilized with saponin (Sigma), and incubated with PE-labeled anti-human IL-2 (BD/PharMingen), anti-human IFN-γ (Caltag Laboratories, Burlingame, Calif.) or anti-human IL-4 (Caltag Laboratories) antibodies. Specific staining is shown on live Vα24⁺ cells.

### Example 2

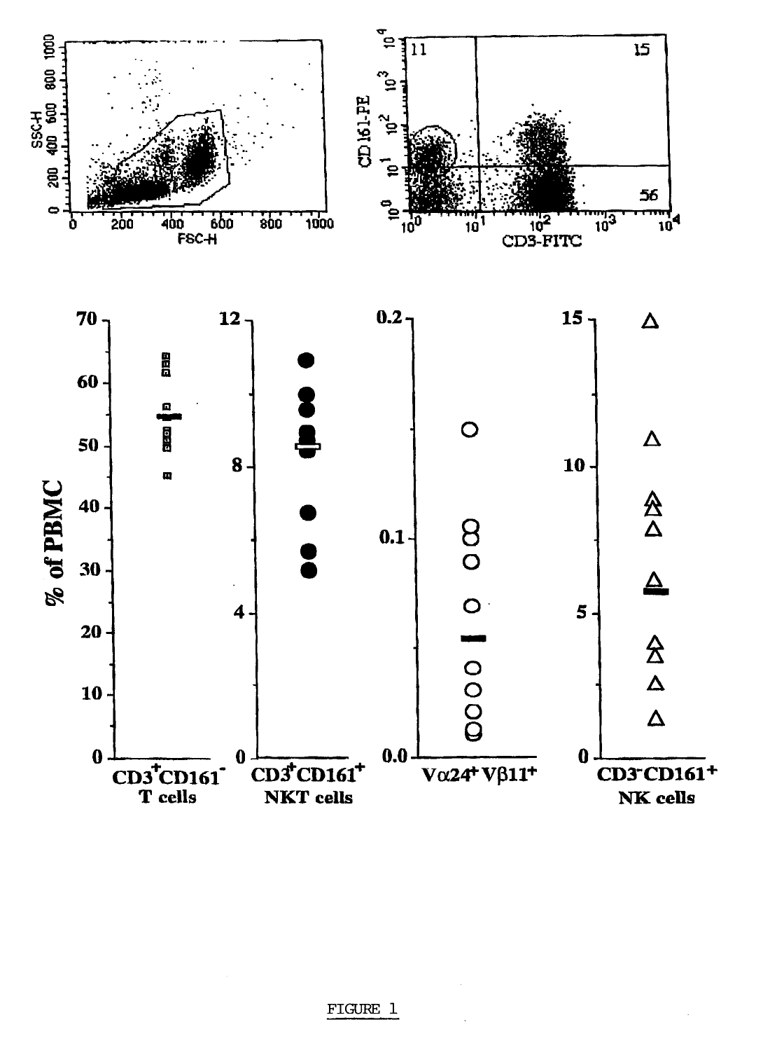

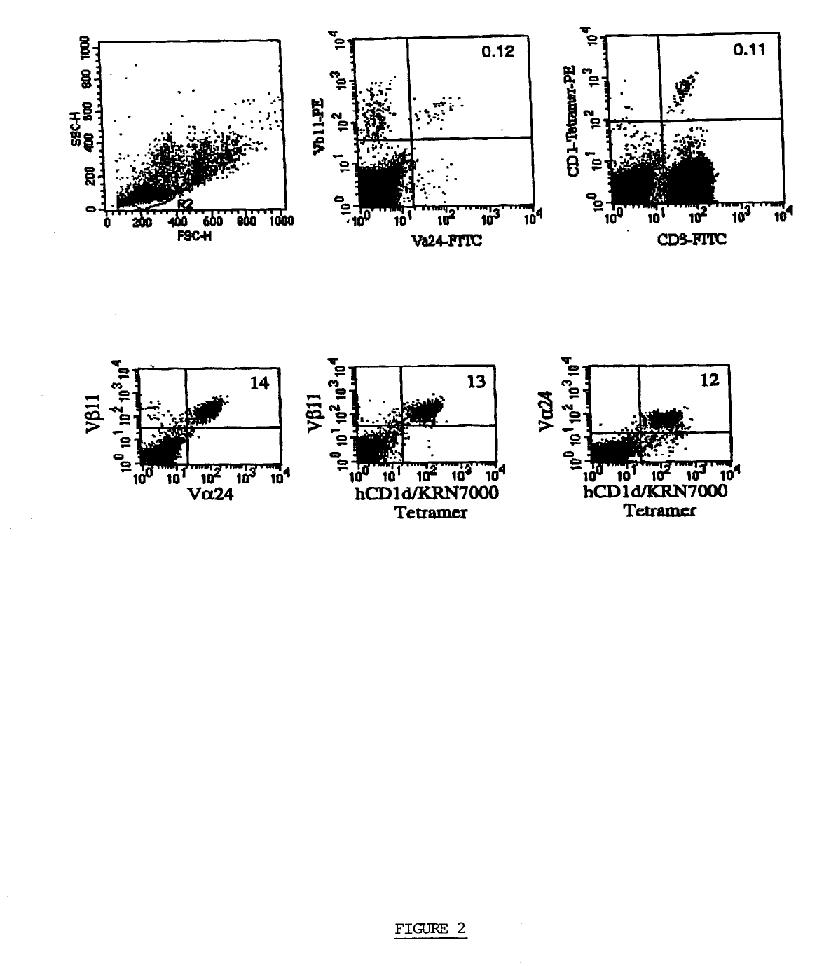

This example describes data indicating that there are NKT cells in healthy human donor PBMC having a Vα24Vβ11 T cell receptor, and that these cells bind human hCD1d-KRN7000-tetramer reagent. Human PBMC contains many different cell types, some of which can be distinguished with antibodies to CD3 and CD161. Conventional T cells (CD3⁺CD 161⁻) comprise the majority of cells (average 55%) in donor PBMC whereas NK cells (CD3⁻CD161⁺) comprise about 2-15% (average 6; FIG. 1). Ten to twenty percent of CD3⁺ T cells express the CD161 surface marker and are classified as NKT cells Bendelac, et al., Annu Rev Immunol 15:535 (1997). These cells represent 5-11% of cells in PBMC. A very small percentage of T cells (0.006-0.15%) express the Vα24Vβ11 T cell receptor (FIG. 1, bottom panel and FIG. 2, top panel) and show variable expression of NK cell markers such as CD94, CD161, NKG2A, and 2B4 (Exley, et al., J Exp Med 186:109 (1997); Wilson, et al., Nature 391:177 (1998); Lee, et al., J. Exp Med 195:637 (2002)).

Donor PBMC contains a small percentage of T cells which express a Vα24/Vβ11 T cell receptor (FIG. 1). Approximately the same percentage of cells in PBMC is also identified by antibody to CD3 and a novel human CD1d-tetramer reagent (hCD1d/KRN7000 Tetramer) which is loaded with KRN7000 (Kroft and Swain, J Immunol 154:4269 (1995); Benlagha, et al., J Exp Med 191:1895 (2000), as shown in FIG. 2 (top panel). The frequency of Vα24⁺Vβ11⁺ cells in healthy donor PBMC is approximately the same as hCD1d/KRN7000-Tetramer⁺/CD3⁺ cells (Gumperz, et al., J Exp Med 195:625 (2002)). These data support the hypothesis that hCD1d-KRN7000 Tetramer binds to and identifies the Vα24⁺Vβ11⁺ T cell receptor-positive population of T cells.

Addition of KRN7000 and rhIL-2 to PBMCs induces expansion of a particular T cell subset which expresses a Vα24/Vβ11 T cell receptor. These cells can be identified with antibodies to Vα24 and Vβ11 or with hCD1d-KRN7000-tetramer plus anti-CD3 antibody (Gumperz, et al., J Exp Med 195:625 (2002)).

The data indicate that the T cells which expand in vitro in response to KRN7000 are nearly all Vα24⁺Vβ11⁺ and CD1d/KRN7000 Tetramer⁺ (FIG. 2, bottom panel). After a 7 day culture with KRN7000 and rhIL-2, 14% of the live cells in the human PBMC are stained with antibodies to Vα24 and Vβ11 (FIG. 2, bottom panel). hCD1d/KRN7000 Tetramer binds approximately the same percentage of cells and nearly all hCD1d/KRN7000 Tetramer⁺ cells are also positive of Vα24 and Vβ11. These data show that the cells which expand in response to KRN7000 can be identified with either antibodies to the T cell receptor chains (Vα24 and Vβ11) or with hCD1d/KRN7000 Tetramer reagent. Nearly all hCD1d/KRN7000 Tetramer-binding cells are Vα24⁺. Therefore, hCD1d-KRN7000-tetramer (with or without anti-CD3 antibody) can be used interchangeably with antibodies to Vα24 and Vβ11 to identify cells which expand in response to KRN7000.

### Example 3

This example describes data indicating that exogenous growth factor can expand KRN7000-reactive (Vα24⁺Vβ11⁺) T cells.

As shown in Table 1, stimulation of healthy donor PBMC with KRN7000 alone or IL-2 alone induces expansion of very few hCD1d-KRN7000-tetramer⁺CD3⁺ T cells (both in percentage and cell number). However, addition of cell survival factors such as IL-2 or IL-15, can greatly increase both the percentage and number of Tetramer⁺ T cells in culture. Compared to KRN7000 alone, addition of IL-2 with KRN7000 increased the number of Tetramer⁺ T cells in culture approximately 300-fold. Similar data was seen in more than 8 different donors.

TABLE 1
Expansion of CD3⁺hCD1d/KRN7000 Tetramer⁺ T cells

| | % CD³⁺ | cell recovery × | number of |
|---|---|---|---|
| Sample | Tetramer⁺ cells | 10⁶ | Tetramer⁺ cells |
| KRN7000 only | 0.03 | 1.04 | 312 |
| IL-2 only | 0.28 | 0.71 | 1,988 |
| KRN7000 ⁺ IL-2 | 8.9 | 1.05 | 93,450 |
| KRN7000 ⁺ IL-15 | 7.2 | 0.66 | 47,520 |

PBMC (1×10⁶/ml) were cultured with KRN7000 alone (100 ng/ml), rhIL-2 alone, or with 100 ng/ml of KRN7000 plus 10 ng/ml rhIL-2 or 100 ng/ml rhIL-15 for 7 days. Percentage of CD3⁺Tetramer⁺ cells was determined by flow cytometry using antibody to CD3 and hCD1d/KRN7000 Tetramer. Cell recovery was determined by trypan blue exclusion. The number of Tetramer⁺ cells was determined by multiplying the percentage of CD3 ⁺Tetramer⁺ cells by the number of cells recovered.

A summary of Vα24⁺Vβ11⁺ T cell expansion from 20 donor PBMC stimulated with 7000 and rhIL-2 is shown in Table 2. After 7 days of culture with KRN7000 and rhIL-2, percentage of Vα24⁺Vβ11⁺ T cells in culture increased an average of 54-fold (range 7-186-fold).

TABLE 2
Vα24⁺Vβ11 T cell expansion from PBMC

| | percentage of Va24⁺Vβ11⁺ cells | | Fold expansion of |
|---|---|---|---|
| Donor | day 0 | day 7 | Vα24⁺Vβ11⁺ cells. % |
| 2.11 | 0.20 | 19 | 95 |
| 1 | 0.11 | 1.8 | 16 |
| 2 | 0.04 | 6.6 | 165 |
| 3 | 0.008 | 0.12 | 15 |
| 4 | 0.59 | 23 | 39 |
| 5 | 0.07 | 13 | 186 |
| 8 | 0.03 | 0.20 | 7 |
| 9 | 0.05 | 1.5 | 30 |
| 10 | 0.08 | 1.1 | 14 |
| 11 | 0.04 | 0.33 | 8 |
| 12 | 0.10 | 3.6 | 36 |
| 13 | 0.008 | 0.40 | 50 |
| 14 | 0.04 | 2.3 | 58 |
| 15 | 0.02 | 0.72 | 36 |
| 16 | 0.006 | 0.37 | 62 |
| 17 | 0.10 | 2.7 | 27 |
| 18 | 0.09 | 12 | 33 |
| 19 | 0.07 | 5.3 | 76 |
| 20 | 0.07 | 0.56 | 8 |
| 21 | 0.07 | 0.89 | 13 |

The percentage of Vα24⁺Vβ11⁺ cells in PBMC (day 0) or PBMC cultured for 7 days with ng/ml KRN7000 and 10 ng/ml rhIL-2 was determined by flow cytometry. The fold expansion in the percentage of Vα24⁺Vβ11⁺ cells after 7 days in culture is shown in right column.

### Example 4

This example describes data indicating that exogenous IL-2 can be substituted with addition of tetanus toxoid.

The use of exogenous IL-2 to expand T cells in vitro and in vivo results in both specific expansion of the cells of interest but also expands other populations of T cells. If IL-2 or other cell survival factors can be generated in situ in sufficient amounts, then the amount of exogenous IL-2 may be reduced or eliminated. The need for exogenous IL-2 (in expansion of Vα24⁺Vβ11⁺ cells stimulated with KRN7000) may be overcome by addition of proteins to which the donor has been immunized.

Since many humans have been immunized with diphtheria/pertussis/tetanus (DPT) and boosted with tetanus toxoid, they contain memory T cells which become activated and secrete cytokines in response to tetanus toxoid. PBMCs respond to tetanus toxin. Addition of tetanus toxoid to healthy donor PBMC induces cell proliferation as measured by tritiated thymidine incorporation (Table 3, right-hand column). In contrast, addition of KRN7000 alone induced little or no proliferation of cells. The addition of tetanus toxoid to KRN7000 cultures resulted in increased cell proliferation over that of KRN7000 or tetanus toxoid alone. Addition of IL-2 to KRN7000 cultures resulted in much higher cell proliferation; however, much of this increase in proliferation was due to IL-2 alone. The most dramatic affect of tetanus toxoid is in the number of Vα24/Vβ11 cells which can be recovered on day 7 (third column). Culture with KRN7000, IL-2, or tetanus toxoid alone resulted in little expansion of Vα24⁺Vβ11⁺ cells; however, addition of tetanus toxoid with KRN7000 greatly increased numbers of Vα24⁺Vβ⁺ cells. The number of T cells generated with tetanus toxoid plus KRN7000 was 65% that of cultures which received exogenous IL-2 in addition to KRN7000 (32,000 v. 49,580). Therefore, tetanus toxoid could partially substitute for the cell proliferative effects of exogenous IL-2 and may be used as agent to assist Vα24⁺ T cell expansion. Similar results were seen in 3 of 4 other donors.

TABLE 3
Tetanus toxoid (TT) can partially replace exogenous IL-2 in
Vα24⁺ Vβ11⁺ cell expansion

| | Cell recovery × | Percentage of | Number of | Proliferation, |
|---|---|---|---|---|
| Condition | 10⁶ | Vα24/Vβ11 cells | Vα24Vβ11 cells | cpm |
| Before culture | 1.0 | 0.07% | 700 | |
| IL-2 ⁺ DMSO | 1.01 | 0.20 | 2,020 | 30,233 |
| KRN7000 only | 0.85 | 0.25 | 2,125 | 1,145 |
| TT only | 1.2 | 0.60 | 7,200 | 11,783 |
| TT ⁺ KRN7000 | 1.28 | 2.5 | 32,000 | 16,431 |
| KRN7000 ⁺ IL-2 | 1.34 | 3.7 | 49,580 | 45,704 |
| TT ⁺ KRN7000 ⁺ IL-2 | 1.72 | 3.1 | 53,320 | 51,405 |

Right column (proliferation) shows tritiated thymidine incorporation of cells cultured for 5 days. One of 5 representative donors is shown. PBMC (1×10⁶/ml) from donor 9 were cultured for 7 days with 100 ng/ml KRN7000, tetanus toxoid (TT), 10 ng/ml rhIL-2 (IL-2), or vehicle (DMSO). Cell recovery was determined by trypan blue exclusion and the percentage of Vα24⁺Vβ11⁺ cells was determined by flow cytometry. The number of Vα24⁺Vβ11⁺ cells was determined by multiplying the cell recovery by the percentage of Vα24⁺Vβ11⁺ cells in culture.

### Example 5

This example describes data indicating that use of donor serum can augment expansion of Vα24/Vβ11 T cells.

The use of donor-specific or autologous serum was best for T cell expansion. Table 4 shows that the use of heat-inactivated autologous donor serum can augment the expansion of Vα24/Vβ11 cells in vitro versus cultures receiving commercial pooled human serum or human AB serum. In 3 donors, the increase in Vα24/Vβ11 cells expanded with KRN7000 and donor autologous donor serum was up to 7 times greater than with commercial pooled human serum. These results indicate that autologous human serum can increase the number of T cells expanded with KRN7000 and is likely to provide the greatest proliferative effect for the initial culture of PBMCs.

TABLE 4
Donor serum provides optimal expansion of Vα24⁺Vβ11⁺ T cells

| | Fold increase in Vα24⁺Vβ11⁺ | | |
|---|---|---|---|
| | T cell number over 7 days | | |
| | Pooled | human | donor |
| Donor PBMC | Human Serum | AB serum | serum |
| 13 | 3.4 | 11 | 25 |
| 14 | 68 | 243 | 395 |
| 15 | 11 | 67 | 73 |

The fold increase in Vα24⁺Vβ11⁺ cells after 7 days of culture is shown. One of two experiments is shown with similar data seen with 4 other donors. PBMCs (1×10⁶/ml) from donors 13-15 were cultured with KRN7000 (100 ng/ml) and rhIL-2 (10 ng/ml) for 7 days in medium containing 10% pooled serum, 10% AB serum, or 10% donor serum. Cells were counted and the percentage of Vα24⁺Vβ11⁺ cells was determined by flow cytometry. The number of Vα24⁺Vβ11⁺ cells before and after culture was determined by multiplying the number of cells recovered by percentage of Vα24⁺Vβ11⁺ cells in culture (determined by flow cytometry).

### Example 6

This example describes data indicating that CD3⁺ Tetramer⁺ (Vα24⁺Vβ11⁺) T cells can be expanded in vitro by repeated culturing (stimulation) with KRN7000 and IL-2, and that healthy donor NKT cell lines continue to expand in vitro after sorting and restimulation.

As indicated in Tables 1 and 2, initial stimulation of PBMC with KRN7000 and rhIL-2 results in tremendous expansion of Vα24⁺Vβ11⁺ (or CD3⁺ Tetramer⁺) cells after 7 days. This expansion continues when cultures are restimulated with KRN7000-pulsed, irradiated, autologous PBMC and rhIL-2 (Tables 5-7). In Tables 5 and 6, the T cells are initially expanded for 7 days with KRN7000 and IL-2. An aliquot of cells is then restimulated with PBMCs from the same donor (that is autologous PBMCs). The use of whole unseparated autologous PBMCs is simple and avoids the need to purify antigen presenting cells for T cell restimulation and expansion.

Results from the restimulation of 9 donor cultures (Table 5) shows that the percentage of Vα24⁺Vβ11⁺ cells continues to expand in most cultures after each restimulation period (on day 7, 14, and 21). After 21-28 days of culture (2-3 rounds of restimulation), the majority of cells in 4 of 9 donors had a Vα24/Vβ11 phenotype. Using this restimulation method, no sorting of Vα24⁺ T cells is needed in order to obtain high numbers of relatively pure T cells (Vα24⁺). Quantitation of Vα24⁺Vβ11⁺ T cell numbers from these cultures showed that there was a 2-5 million-fold expansion of T cells within 35 days of culture in 3 donors (Table 6). Expansion of Vα24⁺Vβ11⁺ T cells was less in one donor (#16), only about 1000-fold in 4 weeks. The fold increase in Vα24⁺Vβ11⁺ cell number was greatest in the first 7 days and generally slowed after repeated stimulations (Table 6).

As shown in Tables 5 and 6, restimulation of PBMC cultures with KRN7000-pulsed autologous PBMCs results in the increase of Vα24⁺Vβ11⁺ cells over time. However, in some donors, the percentage of Vα24⁺Vβ11⁺ cells remained low or tended to decrease upon repeated stimulation (Table 5, donors 13, 16, and 18).

In order to study the function of the expanded NKT cells and their potential for long-term expansion in vitro, short-term cultures (1-2 weeks) of PBMC, stimulated with KRN7000 and IL-2, were sorted with antibody to the Vα24 T cell receptor or with anti-Vα24 antibody plus anti-CD4. The data indicate that culture and restimulation of human Vα24⁺ T cell lines can be extended for more than 3 weeks. Table 7 shows expansion of two Vα24⁺-sorted human T cell lines that were restimulated discontinuously for up to 11 weeks. Stimulation of T cell lines with IL-2 and irradiated, allogeneic, KRN7000-pulsed PBMCs induced T cells to expand an average of 8-fold per week. Calculation of the total T cell expansion over 10 weeks showed that NKT cells could be expanded at least one billion-fold (10⁹). When the initial expansion of the pre-sorted Vα24⁺ cells from the initial PBMC samples is included (Table 2), then the total expansion potential of these cells is approximately 10¹¹.

TABLE 5
Percentage of Vα24⁺Vβ11⁺ cells
in culture after restimulation

% of Vα24⁺Vβ11⁺ cells in culture

| Donor | day 0 | day 7 | day 14 | day 21 | day 28 |
|---|---|---|---|---|---|
| 13 | .008 | .47 | 1.1 | 1 | |
| 14 | .015 | 6.3 | 33 | 55 | |
| 15 | .025 | 1.9 | 17 | 44 | |
| 16 | .006 | 0.28 | 1.5 | 2.8 | 0.8 |
| 17 | .10 | 20 | 44 | 75 | 78 |
| 18 | .09 | 11 | 38 | 18 | 7.6 |
| 19 | .07 | 11 | 66 | 83 | 80 |
| 20 | .083 | 0.6 | 3.5 | 19 | 28 |
| 21 | .053 | 0.5 | 2.3 | 41 | 62 |

Donor PBMC (1×10⁶/ml) were stimulated with 100 ng/ml KRN7000 and 10 ng/ml rhIL-2 for 7 days. An aliquot of cells (0.2-1×10⁶) was then restimulated with 3-5 times as many KRN7000-pulsed irradiated autologous PBMCs and rhIL-2 every 7 days. After each culture period, the percentage of Vα24⁺Vβ11⁺ cells was determined by flow cytometry. Day 0 represents cells in PBMC ex vivo with no culture.

TABLE 6
Expansion in % of Vα24Vβ11⁺ cells
in culture T cell numbers after restimulation

| | Fold expansion in Vα24⁺ Vβ11⁺ cell number | | | | | |
|---|---|---|---|---|---|---|
| Donor | day 7 | day 14 | day 21 | day 28 | day 35 | Total expansion |
| 16 | 11 | 18 | 6.3 | 1.3 | NA | 1.6 × 10³ |
| 17 | 340 | 13 | 18 | 17 | 5.3 | 3 × 10⁶ |
| 18 | 193 | 29 | 5 | 3.6 | 24 | 2.4 × 10⁶ |
| 19 | 178 | 39 | 23 | 6.3 | 4.3 | 5.6 × 10⁶ |

Healthy donor PBMCs (1×10⁶/ml) were cultured and restimulated weekly as in Table 5 for up to 35 days. After each round of stimulation, cells were harvested, counted, and the percentage of Vα24⁺Vβ11⁺ cells was determined by flow cytometry. The number of Vα24⁺Vβ11⁺ cells present in culture was determined by multiplying the number of cells recovered by the percentage of Vα24⁺Vβ11⁺ cells in culture. The fold increase in Vα24⁺Vβ11⁺ cell number was determined by dividing the number of cells obtained by the number of cells present 7 previously. The total expansion (right column) represents the actual expansion capacity of Vα24⁺Vβ11⁺ cells after 28-35 days and is derived by multiplying together the expansion seen each week. NA, not assayed.

TABLE 7
Sorted Vα24⁺ human T cell lines can be
expanded in vitro to 10⁹ cells in 10 weeks

Fold expansion in T cell
number per week

| Week of culture | Donor 18 | Donor 20 |
|---|---|---|
| 1 | 8.5 | 4.2 |
| 2 | 8.3 | 6.8 |
| 3 | 5.3 | 11.8 |
| 4 | 6 | 17 |
| 5 | 6.6 | 9.1 |
| 6 | 6.6 | 10.5 |
| 7 | 8.2 | 3.2 |
| 8 | 8.4 | 14.7 |
| 9 | 8.8 | 8.5 |
| 10 | 9.9 | 3.7 |
| 11 | NA | 5.1 |
| average expansion/week | 7.7 | 8.6 |
| total expansion | 5.9 × 10⁸ | 4.2 × 10⁹ |

Vα24 sorted T cell lines (Donor 18 and Donor 20) were stimulated weekly with KRN7000-pulsed, irradiated, allogeneic PBMC and 10 ng/ml rhIL-2 (2×0⁵/ml T cells plus 1×10⁶/ml PBMC). Cells were counted by trypan blue exclusion and fold expansion per week was determined. Cell lines remained greater than 90% Vα24⁺ throughout the culture period (data not shown).

### Example 7

This example describes data indicating that Vα24⁺ T cell lines expanded with KRN7000 and IL-2 secrete large quantities of cytokines upon activation and are cytotoxic and can indirectly activate NK cell cytotoxicity.

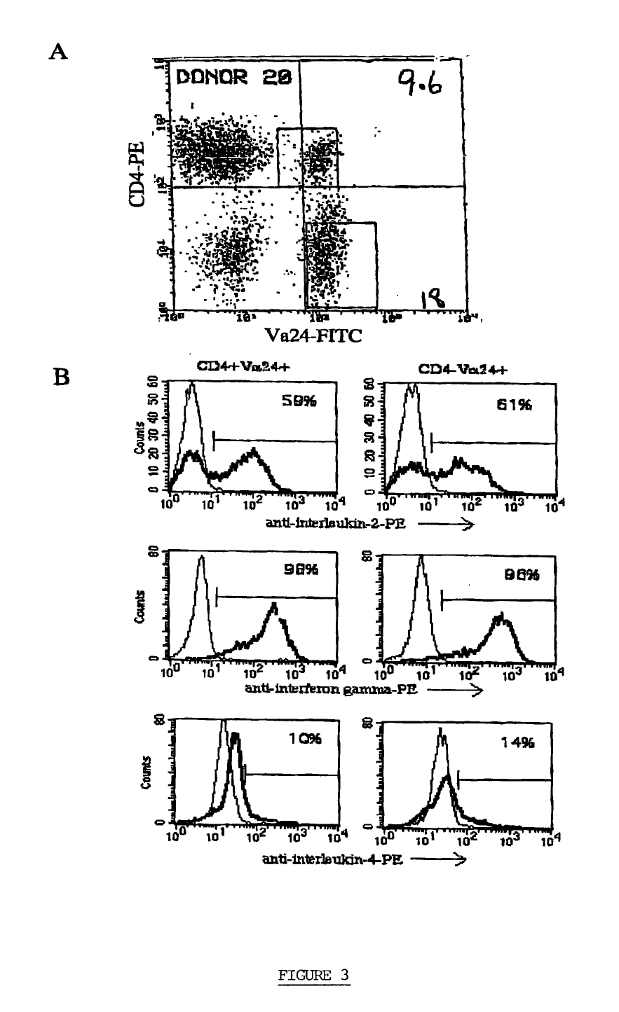

In order to examine the function of expanded Vα24⁺ T cells, Vα24⁺CD4⁺ and Vα24⁺CD4− T cells were purified by fluorescence-activated cell sorting (FACS) (FIG. 3A). These two populations of cells were then expanded for 7 days in vitro with KRN7000-pulsed PBMC and rhIL-2, as in Table 7. The two populations of cells were then stimulated with PMA plus ionomycin in order to determine their cytokine-secretion potential. Results in FIG. 3B (intracellular cytokine staining) show that both T cell populations can produce IL-2, and IFN-γ. IL-4 was detected at low levels in the CD4⁺ subset only (FIG. 3B, lower left histogram).

ELISA analysis of seven different secreted cytokines from 8 different Vα24-sorted NKT cell lines is shown in Table 8. After stimulation with PMA plus ionomycin, both CD4⁺ and CD4⁻ cell lines secreted large quantities of GM-CSF, TNFα, and IFN-γ. Lower and variable levels of IL-2, IL-4, IL-5, and IL-10 are also made. There were no clear differences between CD4⁺ and CD4⁻ lines except in IL-4 secretion. In agreement with published reports (Wilson, et al., Nature 391:177 (1998); Lee, et al., J. Exp Med 195:637 (2002)), the CD4⁺ NKT cells tended to secrete more IL-4 than CD4⁻ NKT cells. Activation of Vα24⁺ T cell lines with KRN7000-pulsed PBMCs also resulted in cytokine secretion, though the levels of IL-2 and GM-CSF produced were 10-50 times less than with PMA plus ionomycin stimulation.

TABLE 8
Cytokine secretion from human NKT cell lines

| | ng/ml of cytokine | | | | | | |
|---|---|---|---|---|---|---|---|
| A. | | | | | B. | | |
| Cell line | IL-2 | IL-4 | IL-10 | IFN-γ | IL-S | TNFα | GM-CSF |
| 4, CD4⁺ | 15 | 13 | .77 | 24 | 10 | 24 | 163 |
| 4, CD4⁻ | 6.6 | 6.6 | .61 | 24 | 7.4 | 15 | 97 |
| 15, CD4⁺ | 6.6 | 3.1 | .02 | 16 | 1.1 | 12 | 97 |
| 15, CD4⁻ | .59 | .03 | .02 | 6.7 | .49 | 2.8 | 30 |
| 17, CD4⁺ | 13 | 3.4 | .02 | 22 | .98 | 14 | 79 |
| 18, CD4⁺ | 21 | 5.6 | .17 | 22 | 2.1 | 35 | 158 |
| 20, CD4⁺ | 1.2 | 1.1 | .09 | 15 | .23 | 8.3 | 77 |
| 20, CD4⁻ | 5 | .03 | .02 | 17 | .09 | 22 | 105 |

Purified CD4⁺ and CD4⁻ Vα24⁺Vβ11⁺ human T cell lines (5×105/ml) from Donors 4, 15, 17, 18, and 20 were stimulated for 24 hours with 10 ng/ml of PMA and 500 ng/ml of ionomycin. Supernatant was collected and cytokine content was determined by ELISA.

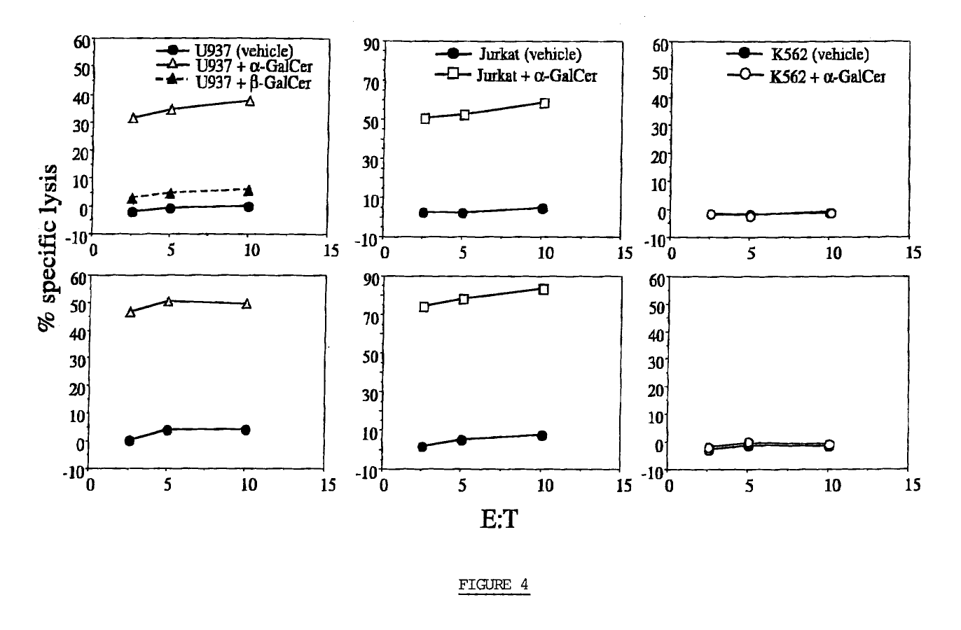

In addition to cytokine secretion, both CD4⁺ and CD4⁻ populations of expanded Vα24⁺ T cells could specifically kill tumor target cell lines which were pulsed with KRN7000 (FIG. 4). Both monocytic (U937) and T cell leukemic target cells (Jurkat) were lysed, but only when they were pre-pulsed with KRN7000. The erythroleukemic cell line, K562, was not lysed even when pre-pulsed with KRN7000 (RAJI and Molt-4 lines were not killed either, data not shown). This pattern of killing correlated with the expression of CD1d on target cells (Metelitsa, et al., J Immunol 167:3114 (2001)) and required the presence of KRN7000. In addition, target cell lysis was very specific for the “α” form of galactosylceramide (KRN7000) because target cells pulsed with the “β” form of galactosylceramide or with vehicle alone (DMSO) were not lysed.

The data in FIG. 4 is corroborated by results from 4 different NKT cell lines from 2 different donors (Table 9). Even at the low effector to target (E:T) ratio of 1.25:1, there is substantial killing (greater than 25%) of KRN7000-pulsed Jurkat tumor cells. In addition, KRN7000-pulsed U937 but not Molt-4 or RAJI cells were killed (data not shown). These results show that the NKT cell lines expanded in vitro by repeated culturing (restimulation) with PBMCs and KRN7000 are functional and kill only in an antigen-dependent (KRN7000) and CD1d-dependent manner.

TABLE 9
Human Vα24⁺ T cell lines can kill KRN7000-pulsed tumor cells

% Specific Lysis
Effector:target ratio

| Cell Line | 1.25:1 | 5:1 |
|---|---|---|
| Donor 4, CD4⁺ | 29 | 45 |
| Donor 4, CD4⁻ | 49 | 61 |
| Donor 15, CD4⁺ | 25 | 37 |
| Donor 15, CD4⁻ | 54 | 63 |
| Donor 20, CD4⁺ | 26 | 41 |
| Donor 20, CD4⁻ | 26 | 41 |

Cultures were set up in triplicate as in FIG. 4 with KRN7000-pulsed, 51 Cr-labeled Jurkat tumor cells. Human Vα24⁺ T cells from 3 different donors were added to Jurkat targets at effector:target (E:T) ratios of 1.25:1 and 5:1. Percent specific lysis is shown. Data is representative of 2 or 3 experiments with each NKT cell line. In the absence of KRN7000, Jurkat targets were not lysed (less than 5% specific lysis).

Previous reports in murine tumor models treated with KRN7000 (Camaud, et al., J Immunol 163:4647 (1999); Jacobson, Pilaro and Smith Proc Natl Acad Sci USA 93:10405 (1996)) and in vitro-derived human NKT cells (Metelitsa, et al., J Immunol 167:3114 (2001)) have shown that killing of tumor cells can be mediated by NK cells. Data in Table 10 indicate that supernatant from activated NKT cells could induce activation and killing activity in NK cells. Although fresh NK cells were not cytotoxic NK cells cultured with supernatant from an activated Vα24 NKT cell line could become activated and efficiently kill tumor cell lines Jurkat, U937, K562, and RAJI (Table 10). Supernatants from two other NKT cell lines were also effective in activating NK cells to kill tumor cells. NK cells activated with NKT cell supernatant were nearly identical in activity to NK cells activated with recombinant IL-2 plus IFN-γ, cytokines shown to be important for NK cell cytotoxic activity (Metelitsa, et al., J Immunol 167:3114 (2001)). However, unlike killing by NKT cells (FIG. 4 and Table 9), killing by NK cells did not require KRN7000 to be presented by the tumor target cell. Therefore, activation of NKT cells in vivo may result in tumor cell lysis by a direct mechanism (NKT cell killing of CD1d⁺ and tumor cells) and by an indirect mechanism (NK cell activation and killing of both CD1d⁺ and CD1d⁻ tumor cells).

TABLE 10
Vα24⁺ T cells indirectly activate NK cell cytotoxicity.

| | % Specific Lysis at 5:1 E:T ratio | | | |
|---|---|---|---|---|
| Effector cells | Jurkat | U937 | K562 | RAJI |
| Fresh NK cells | 2.6 | 0.6 | 7.3 | 0.6 |
| NKT-supematant treated | 44 | 35 | 53 | 38 |
| IL-2 ⁺ IFN-γ-treated | 46 | 33 | 43 | 31 |

Fresh human CD56⁺ NK cells were cultured with supernatant from an activated CD4⁺ NKT cell line (Donor 15, CD4⁺) or with 2 ng/ml of rhIL-2 and 10 ng/ml of rhIFN-γ for 3 days prior to use as effectors in a chromium release assay. Killing activity of cultured NK cells was compared with fresh NK cells in a chromium release assay as described in FIG. 4 (except that effector cells are NK cells, not T cells). Various numbers of effector NK cells were cultured with ⁵¹Cr-labeled target cells (5×10³/well) in triplicate. Specific lysis from averaged counts is shown. Supernatant from the activated Vα24⁺ T contained 8.7 ng/ml IL-2, 10 ng/ml IL-4, and 28 ng/ml IFN-γ determined by ELISA) was used at a 1:2 dilution for pre-incubation of NK cells.

What is claimed is:

1. A method for stimulating the proliferation of human T cells in vitro comprising repeatedly culturing donor T cells in the presence of antigen presenting cells, an antigen, a cell survival factor and serum for at least 7 days under conditions stimulating proliferation of the T cells.

2. The method of claim 1, wherein at least a portion of the proliferated T cells express Vα24 T cell receptor (TCR).

3. The method of claim 1, wherein at least a portion of the proliferated T cells express CD3 or CD161.

4. The method of claim 1, wherein at least a portion of the proliferated T cells are capable of killing a cell

5. The method of claim 1, wherein the antigen presenting cells are from the same human as the human donor T cells or are from a different human.

6. The method of claim 1, wherein the antigen presenting cells are human or are non-human.

7. The method of claim 1, wherein the antigen presenting cells are engineered to express human or non-human CD1a, CD1b, CD1c or CD1d, or a molecule having the glycolipid binding activity of human or non-human CD1a, CD1b, CD1c or CD1d.

8. The method of claim 1, wherein the antigen presenting cells are present in or obtained from human peripheral blood mononuclear cells (PBMC).

9. The method of claim 1, wherein the cells are passaged at least five times.

10. The method of claim 1, wherein the serum is human.

11. The method of claim 1, wherein the serum is non-human.

12. The method of claim 1, wherein the serum is replaced with a serum-free medium.

13. The method of claim 1, wherein the cell survival factor comprises a molecule that binds to a molecule on the surface of a T cell.

14. The method of claim 1, wherein the cell survival factor comprises IL-2.

15. The method of claim 1, wherein the cell survival factor comprises IL-2, IL-7 or Il-15.

16. The method of claim 1, wherein the cells proliferate to about 10⁸ cells or greater over 10 weeks.

17. The method of claim 1, wherein the antigen comprises a glycosphingolipid, a bacterial antigen or a viral antigen.

18. The method of claim 17, wherein the glycosphingolipid comprises KRN 7000 or a KRN 7000 analogue.

19. The method of claim 18, wherein the KRN7000 analog comprises β-glucosylceramide (β-GluCer).

20. The method of claim 17, wherein the bacterial antigen comprises tetanus toxoid, diphtheria toxin, BCG, pertussis antigen, Hemophilus influenzae type B antigen or a pneumoccoccol antigen.

21. The method of claim 17, wherein the viral antigen comprises a measles virus antigen, rubella viral antigen, varicella viral antigen, or hepatitis viral antigen.

22. The method of claim 1, wherein the donor T cells are antigen sensitized.

23. The method of claim 1, wherein at least a portion of the proliferated T cells produce one or more cytokines.

24. The method of claim 1, wherein at least a portion of the proliferated T cells exhibit anti-tumor cell activity.

25. The method of claim 1, wherein at least a portion of the proliferated T cells activate NK cells to exhibit anti-tumor cell activity.

26. The method of claim 1, wherein the antigen presenting cells are non-viable.

27. The method of claim 1, wherein the antigen presenting cells are irradiated.

28. A method for stimulating the proliferation of human T cells in vitro comprising repeatedly culturing donor T cells in the presence of antigen presenting cells, an antigen, IL-2, without IL-7 or IL-15, and serum under conditions stimulating proliferation of the T cells.

29. The method of claim 28, wherein at least a portion of the proliferated T cells express Vα24 T cell receptor (TCR).

30. The method of claim 28, wherein at least a portion of the proliferated T cells express CD3 or CD161.

31. The method of claim 28, wherein at least a portion of the proliferated T cells are capable of killing a cell

32. The method of claim 28, wherein the antigen presenting cells are from the same human as the human donor T cells or are from a different human.

33. The method of claim 28, wherein the antigen presenting cells are human or are non-human.

34. The method of claim 28, wherein the antigen presenting cells are engineered to express human or non-human CD1a, CD1b, CD1c or CD1d, or a molecule having the glycolipid binding activity of human or non-human CD1a, CD1b, CD1c or CD1d.

35. The method of claim 28, wherein the antigen presenting cells are present in or obtained from human peripheral blood mononuclear cells (PBMC).

36. The method of claim 28, wherein the cells are passaged at least five times.

37. The method of claim 28, wherein the serum is human.

38. The method of claim 28, wherein the serum is non-human.

39. The method of claim 28, wherein the serum is replaced with a serum-free medium.

40. The method of claim 28, wherein the cells proliferate to about 10⁸ cells or greater over 10 weeks.

41. The method of claim 28, wherein the antigen comprises a glycosphingolipid, a bacterial antigen or a viral antigen.

42. The method of claim 41, wherein the glycosphingolipid comprises KRN 7000 or a KRN 7000 analogue.

43. The method of claim 42, wherein the KRN7000 analog comprises β-glucosylceramide (β-GluCer).

44. The method of claim 42, wherein the bacterial antigen comprises tetanus toxoid, diphtheria toxin, BCG, pertussis antigen, Hemophilus influenzae type B antigen or a pneumoccoccol antigen.

45. The method of claim 42, wherein the viral antigen comprises a measles virus antigen, rubella viral antigen, varicella viral antigen, or hepatitis viral antigen.

46. The method of claim 28, wherein the donor T cells are antigen sensitized.

47. The method of claim 28, wherein at least a portion of the proliferated T cells produce one or more cytokines.

48. The method of claim 28, wherein at least a portion of the proliferated T cells exhibit anti-tumor cell activity.

49. The method of claim 28, wherein at least a portion of the proliferated T cells activate NK cells to exhibit anti-tumor cell activity.

50. The method of claim 28, wherein the antigen presenting cells are non-viable.

51. The method of claim 28, wherein the antigen presenting cells are irradiated.

52. A proliferated T cell culture produced by the method of claims 1 or 28.

53. A method for providing cell therapy to a subject comprising administering to the subject a T cell culture of claim 28 in an amount sufficient to provide therapy to the subject.

54. The method of claim 53, wherein the donor T cells are obtained from the subject to which the proliferated T cells are administered.

55. The method of claim 53, wherein the subject has or is at risk of having undesirable numbers of T cells.

56. The method of claim 53, wherein the subject has or is at risk of having undesirable numbers of T cells that express Vα4 T cell receptor.

57. The method of claim 53, wherein the subject is a candidate for or has undergone organ or tissue transplant.

58. The method of claim 53, wherein the subject has or is at risk of having an immune deficiency, an autoimmune disorder, a cancer, or an infectious disease.

59. The method of claim 58, wherein the immune deficiency is associated with an organ or tissue transplant.

60. The method of claim 58, wherein the autoimmune disorder is selected from: diabetes, multiple sclerosis, systemic sclerosis, colitis, hepatitis, lupus, rheumatoid arthritis or Sjögren's syndrome.

61. The method of claim 58, wherein the cancer comprises a solid tumor, metastatic tumor, leukemia, lymphoma or myeloma.

62. The method of claim 61, wherein the leukemia comprises T cell, B cell or monocytic leukemia.

63. The method of claim 61, wherein the lymphoma comprises Hodgkin's lymphoma or non-Hodgkin's lymphoma.

64. The method of claim 58, wherein the cancer comprises an adenocarcinoma, plasmacytoma, sarcoma, carcinoma or neuroblastoma.

65. The method of claim 58, wherein the infectious disease is caused by a virus, bacterium, fungus, or a parasite.

66. The method of claim 65, wherein the virus comprises human immunodeficiency virus (HIV), hepatitis C virus (HCV), cytomegalovirus or hepatitis B virus (HBV).

67. The method of claim 65, wherein the bacterium causes tuberculosis, lyme disease or leprosy.

68. The method of claim 65, wherein the parasite causes malaria or Chagas' disease.

69. A method for producing a population of proliferated human T cells, comprising repeatedly culturing donor T cells in the presence of antigen presenting cells, an antigen, a cell survival factor and serum for at least 7 days, thereby producing a population of proliferated human T cells.

70. The method of claim 69, wherein the cell survival factor comprises IL-2.

71. The method of claim 69, wherein the cell survival factor comprises IL-2 without IL-7 or IL-15.

72. A method for producing a population of proliferated human T cells, comprising repeatedly culturing donor T cells in the presence of antigen presenting cells, an antigen, IL-2, without IL-7 or IL-15, and serum, thereby producing a population of proliferated human T cells.

73. The method of claim 72, wherein the donor T cells are cultured for at least 7 days.
